(19) United States
(12) Patent Application Publication
(10) Pub. No.: US 2006/0270998 A1
(43) Pub. Date: Nov. 30, 2006

(54) Device transforming the pain of a syringe needle into a tolerable feeling

(76) Inventor: Karim-Frédéric Marti, La Landeron (CH)

Correspondence Address: SUGHRUE MION, PLLC, 2100 PENNSYLVANIA AVENUE, N.W., SUITE 800, WASHINGTON, DC, 20037, US

(21) Appl. No.: 10/534,743

(22) PCT Filed: Nov. 13, 2002
(86) PCT No.: PCT/EP02/12788

§ 371 Date: May 11, 2006

(51) Int. Cl. A61M 5/32

(52) U.S. Cl. 604/198

(57) ABSTRACT

There is disclosed a device taking the form of a sleeve (20) for capping a syringe (1) of a conventional type. The front end (25) of the sleeve (20) has an edge (28) to be applied with a certain amount of pressure onto the skin of the patient to be treated all the way round the point of insertion of the needle (8) of the syringe. The resulting compression of the skin in this area reduces the pain associated with insertion of the needle into the skin.

The present invention concerns a device for reducing the pain associated with insertion of a syringe needle into the skin and, more particularly, a device of this type able to be used in the oral cavity.

Such devices have already been disclosed in the prior art.

International Patent Application No. WO 02/09575, published 7 Feb. 2002, discloses a device for reducing the pain felt by a patient undergoing an injection by syringe at the moment of insertion of the syringe needle into the skin. This device is made in the form of a plate, that is preferably flexible, and comprises, on one of its faces, a plurality of small points intended to be applied onto the patient's skin with a certain amount of pressure. An aperture is provided at the centre of the plate to allow the syringe needle to pass through at moment the injection is carried out. The operation of this device is based on the application of small points in the region of the skin located around the point of injection shortly before the injection is carried out. In this way, the patient's nervous system is disturbed and the pain associated with insertion of the needle into the skin is reduced. This document suggests that it is necessary to provide a surface large enough to make the plate carrying the small points in order to guarantee a significant reduction in the pain associated with insertion of the needle.

This solution has, however, certain drawbacks, particularly significant bulk limiting the use thereof to areas of the patient's body that are easily accessible. By way of example, it is difficult to use this device to carry out an injection into the patient's gums.

Moreover, another drawback of the solution that has just been described is linked to ergonomics. Because of its structure, the plate requires the use of a hand to press the plate homogenously over its entire surface, the second hand being used in such case to hold the syringe and carry out the injection. Such a solution can prove inconvenient for the operator who may sometimes need a free hand to carry out associated operations.

It is an object of the present invention to improve the devices of the prior art so as to avoid the aforecited drawbacks and, particularly, to provide a device that can easily be used with a single hand to carry out injections in the oral cavity.

Therefore, the invention provides a device comprising a syringe fitted with a needle for reducing the pain associated with insertion of the needle into the skin, characterized in that it comprises a capping element, or sleeve, delimiting an inner space able to at least partially receive the syringe and comprising a first aperture in its rear part, through which the syringe is inserted into the inner space, and a second aperture in its front part through which the needle passes when the latter is inserted into the inner space, the front part of the capping element having a projecting portion arranged continuously or discontinuously around the second aperture to form at least one contact zone to be applied against a patient's skin, around a point of injection, prior to insertion of the needle into the skin.

Preferably, the zone of contact with the patient's skin has the shape of an annular edge of small width and is arranged at a short distance around the second aperture. Moreover, since conventional type syringes generally have gripping means with good ergonomics, similar gripping means are provided on the capping element to allow simultaneous handling of the syringe and the sleeve according to the invention using a single hand.

Owing to its structure and compactness, the device according to the present invention allows an operator to carry out an injection at virtually any place on the patient's body, while having one hand free to carry out associated tasks. Thus, to return to the preceding example, an injection by syringe to the patient's gums can easily be carried out while the patient experiences a reduced sensation of pain owing to the invention.

The invention will be better understood with the following description of two embodiment examples referring to the annexed drawings, in which:

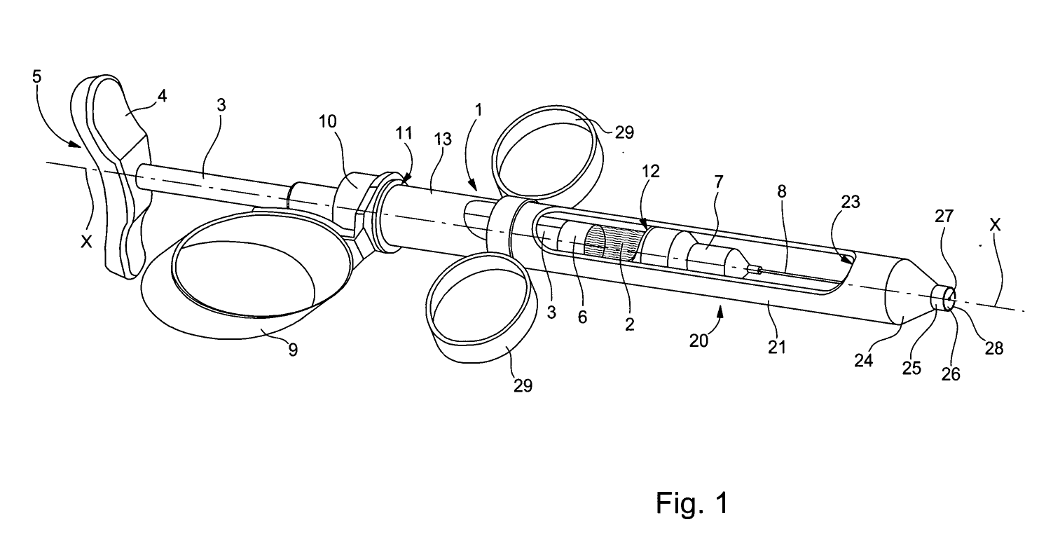

FIG. 1 is a perspective view showing the device according to the present invention, the syringe being partially inserted into the sleeve,

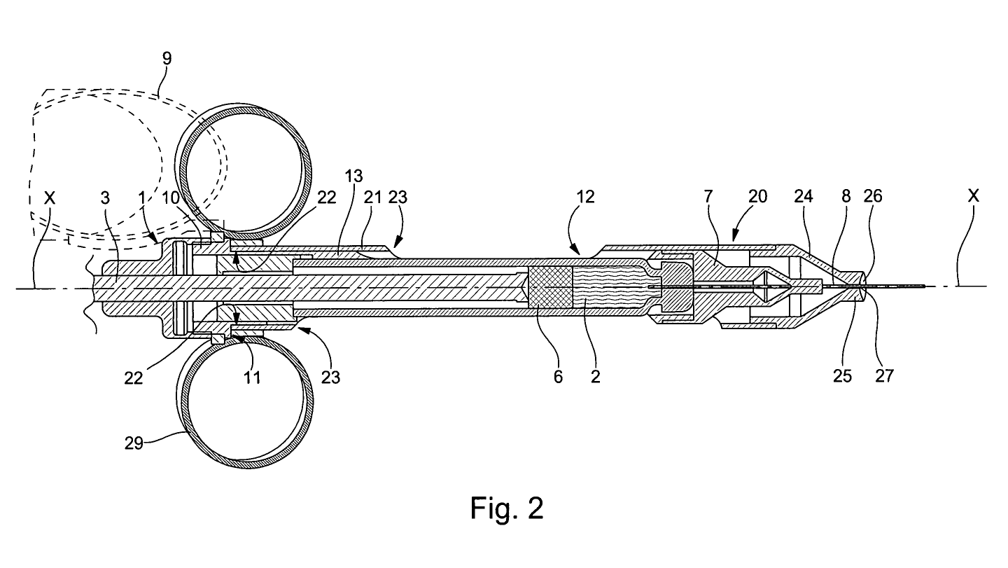

FIG. 2 is a partial cross-section of the device shown in FIG. 1, the syringe being partially inserted into the inner space of the sleeve,

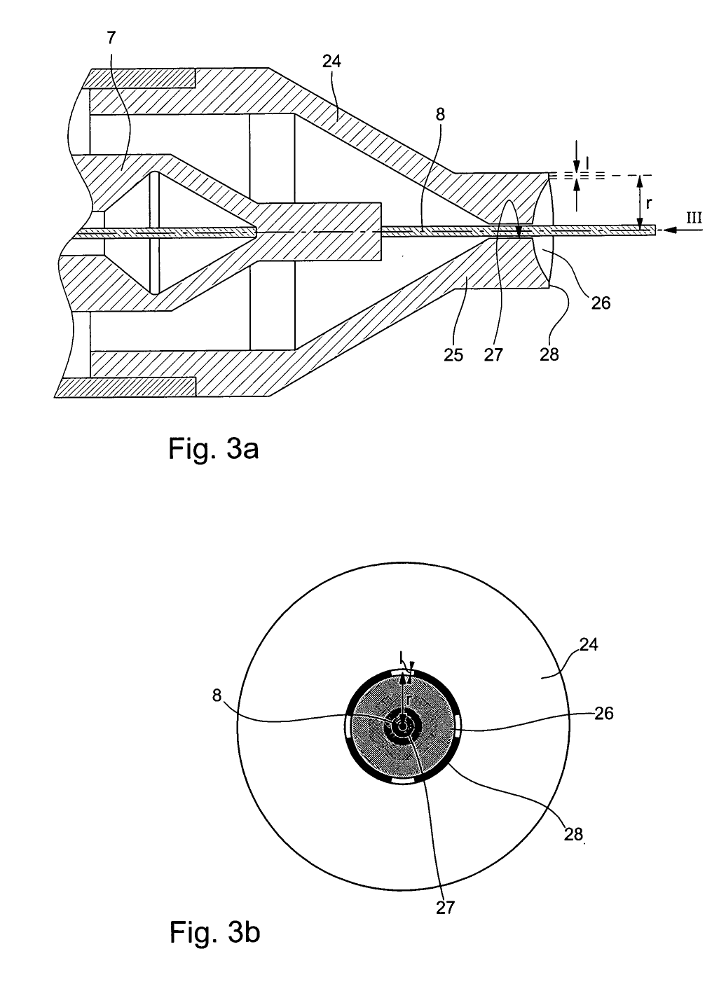

FIG. 3a is an enlarged cross-section of the front part of the sleeve in the same configuration as that shown in FIG. 2;

FIG. 3b is an enlarged schematic view of the front part of the sleeve along the arrow III of FIG. 3a, in accordance with a variant of the contact zone.

In FIGS. 1 and 2, there is shown a syringe 1 of longitudinal axis X, of a conventional type, comprising a chamber 2 for containing a liquid to be injected into a patient. A rod 3, carrying an element 4 having a support surface 5 at its back end, penetrates the interior of chamber 2 of the syringe. Rod 3 also carries a piston 6 at its front end. Piston 6 is made of a slightly elastic material and has an external section that is substantially greater than the internal section of chamber 2, such that it forms a seal between the two parts of the chamber that it delimits. Thus, piston 6 is capable of moving in translation, in the direction of axis X, so as to transmit the pressure exerted by a user on support surface 5 to the liquid located in chamber 2, to eject the latter from syringe 1 when an injection is carried out.

A fitting 7 is connected to the front end of the chamber by a threading or by friction. Fitting 7 carries a needle 8 and, in a conventional manner, enables needles of various dimensions to be fitted to chamber 2.

A gripping element 9 is also provided at the rear end of chamber 2. In the example illustrated, gripping element 9 is ring-shaped, adapted to receive the thumb of the person using the syringe.

It will also be noted that chamber 2 has a short portion 10 with a larger section than its main section so as to form a shoulder 11 in proximity to the rear end of chamber 2. The function of shoulder 11 will be explained hereinafter in relation to FIG. 2.

An observation window 12 has been arranged in an opaque case 13 in which chamber 2 is arranged, to enable the level of liquid to be checked visually when the injection is carried out.

Syringe 1 has been shown in FIG. 1 when it is partially inserted into an inner space delimited by a sleeve or capping element 20. Sleeve 20 has a first main portion 21 of generally cylindrical shape, the inner section of which is substantially greater than the external section of chamber 2 of syringe 1. Sleeve 20 has a circular aperture 22, at its rear end, through which syringe 1 is inserted prior to an injection. Main portion 21 further includes two elongated recesses, or windows, 23 arranged so as to be diametrically opposite and to cooperate with observation window 12 of the syringe, when the latter is in place in the inner space of sleeve 20 (as visible in FIG. 2).

It can be seen in FIG. 3a that sleeve 20 includes, towards its front end, a second portion 24, of truncated shape and ending in an end part 25. The front face 26 of the terminal part in which an opening 27 opens out, has a small surface area. The front face 26, which will be placed in contact with the patient's skin shortly before an injection, includes a projecting portion arranged continuously in the form of an edge 28 around aperture 27. In the example illustrated, which corresponds to a preferred embodiment, a front face 26 has a concave shape between aperture 27 and edge 28. Edge 28 may be continuous or discontinuous and its transverse section may be of semi-circular, square or rectangular shape. FIG. 3b shows a variant in which edge 28 is discontinuous.

According to variants, front face 26 may also include a multitude of raised portions, either concentric, or extending in the radial direction from aperture 27 to edge 28.

Moreover, preferably, the dimensions of edge 28 are chosen such that its width It is comprised between 0.1 and 0.3 millimetres, preferably equal to 0.25 millimetres, and its distance r to second aperture 27 is comprised between 2 and 4 millimetres, preferably equal to 2.5 millimetres.

Thus, within the scope of use in the field of dental care, to return to the aforecited example, it will be observed that the aforementioned dimensions are perfectly suited to carrying out an injection in the vestibule of the oral cavity, i.e. in the area of the mouth where most injections are carried out, this area being particularly difficult to access.

Moreover, sleeve 20 also has gripping means 29 in proximity to its rear end, also made in the form or rings. In a preferred and non-limiting manner, these rings 29 are for receiving respectively the index and second finger of the user of syringe 1.

It will be noted in FIGS. 2 and 3 that, because of their respective dimensions, syringe 1 is liable to slide inside sleeve 20. It will also be noted in this Figure that aperture 27, of small section and arranged at the centre of the front end of sleeve 20, allows needle 8 to pass outside the sleeve.

Thus, syringe 1 and sleeve 20 are shown in this Figure in their configuration adapted to carry out an injection, needle 8 of the syringe having been inserted into the patient's skin.

In this position, shoulder 11 of syringe 1 is arranged in an abutted position against the rear end of sleeve 20, so as to limit the travel of syringe 1 inside sleeve 20. Consequently, shoulder 11 limits the length of needle 8 made to penetrate the patient's skin.

Owing to the structure that has just been described, the device according to the invention advantageously enables a patient to be injected by syringe while reducing the sensation of pain associated with the insertion of needle 8 into the skin.

In fact, an advantageous method of injection by syringe using the device according to the invention comprises the steps consisting in:

applying sleeve 20 via its front part 25, i.e. with the edge 28, on the patient's skin at the desired place for the injection, with a certain amount of pressure and holding it by positioning the index finger and the second finger of the same hand in rings 29,

inserting syringe 1 inside sleeve 20 via aperture 22 of the latter, while controlling its insertion using the thumb of the same hand as that used for holding sleeve 20, the thumb being positioned inside ring 9,

continuing to exert pressure from the thumb against ring 9 when needle 8 reaches the skin of the patient, to insert it into the skin, until shoulder 11 reaches a stop against the rear end of sleeve 20,

exerting pressure on support surface 5 with the palm of the hand to push piston 6 down into chamber 2 and to eject the liquid contained in chamber 2 of syringe 1,

withdrawing the assembly formed by syringe 1 and sleeve 2 by pulling rings 29 using the index and second fingers, so as to extract needle 8 from the patient's skin.

In this way, the pressure applied locally onto the patient's skin by edge 28 of sleeve 20, around the place desired for the injection and prior to the insertion of needle 8 into the skin, reduces the sensation of pain felt by the patient at the moment of the injection. Moreover, as mentioned previously, the device according to the present invention advantageously enables the user of syringe 1 to have one hand free while carrying out the injection owing to the particular arrangement of its gripping means. Furthermore, it will be noted that the needle is therefore protected from any accidental soiling.

Of course, the preceding description describes a preferred embodiment of the present invention in a non-limiting manner and those skilled in the art will be able to carry out secondary alterations without departing from the scope of the invention.

One could envisage, by way of example, using the device according to the present invention for taking blood samples instead of carrying out injections. The structure of sleeve 20 also reduces the pain associated with insertion of the needle into the patient's skin in the case of blood samples.

Likewise, the shape described for the sleeve is not limiting and those skilled in the art could make the later in any other shape that allows the advantageous effects of the invention to be obtained, i.e. the application of the front end of the sleeve must exert compression between the patient's skin all the way around the point of insertion of the needle into the skin. Thus, one could envisage the front face 26 of sleeve 20 and its edge 28 having square or rectangular sections, for example.

Moreover, means (not shown) could also be implemented to position syringe 1 inside sleeve 20, or guiding it during its insertion into the inner space, so as to ensure proper relative positioning of the means for gripping the syringe and the sleeve. Such positioning means, which can be made in the form of a lug cooperating with a groove, for example, further improve the ergonomics of the device according to the present invention, insofar as they prevent any rotation of the syringe in relation to the sleeve during the injection.

1.-17. (canceled)

18. A device including a syringe fitted with a needle for reducing the pain associated with insertion of said needle into the skin, the device including a capping element delimiting an inner space able to at least partially receive the syringe, said capping element including a first aperture arranged in a rear part, through which the syringe can be inserted into said space, and a second aperture arranged in a front part and through which said needle passes when the syringe is inserted into said space, said front part having at least one projecting part arranged continuously or discontinuously around said second aperture to form at least one contact zone or front face to be applied with pressure against a patient's skin, around a point of injection, prior to insertion of the needle into the skin, said syringe including means intended to be pressed by at least one of the user's fingers to insert the needle into the patient's skin, wherein said capping element also has means to be pressed by at least one of the user's fingers to allow the application of such pressure onto the skin of the patient prior to insertion of the needle into the skin, via said front face, said means of the capping element being arranged such that the user can exert pressure simultaneously on said means of the capping element and said means of the syringe using a single hand.

19. A device including a syringe fitted with a needle for reducing the pain associated with insertion of said needle into the skin, wherein said device includes a capping element delimiting an inner space able to at least partially receive the syringe, said capping element including a first aperture arranged in a rear part, through which the syringe can be inserted into said space, and a second aperture arranged in a front part and through which said needle passes when the syringe is inserted into said space, said front part having at least one projecting part arranged continuously or discontinuously around said second aperture and including an edge located at a distance (r) from said second aperture comprised between 2 and 4 millimetres, preferably of the order of 2.5 millimetres, to form at least one contact zone or front face to be applied with pressure against a patient's skin, around a point of injection, prior to insertion of the needle into the skin.

20. The device according to claim 18, wherein said projecting part includes an annular edge.

21. The device according to claim 19, wherein said edge is of annular shape.

22. The device according to claim 20, wherein said edge has a transverse semi-circular section.

23. The device according to claim 20, wherein said edge has a width (I) comprised between 0.1 and 0.3 millimetres, preferably substantially equal to 0.25 millimetres.

24. The device according to claim 20, wherein said edge is arranged at a distance (r) from said second aperture comprised between 2 and 4 millimetres, preferably of the order of 2.5 millimetres.

25. The device according to claim 22, wherein said edge is arranged at a distance (r) from said second aperture comprised between 2 and 4 millimetres, preferably of the order of 2.5 millimetres.

26. The device according to claim 18, wherein said means of the capping element include two rings arranged on either side of the capping element in proximity to said rear part, each of said rings being intended to receive one of the user's fingers other than the thumb.

27. The device according to claim 18, wherein said means of the syringe have substantially the shape of a ring intended to receive the user's thumb.

28. The device according to claim 18, wherein means are provided for forming a stop member during insertion of the syringe into said inner space of the capping element.

29. The device according to claim 20, wherein said front face of said capping element extends from said second aperture to said edge along a concave shape.

30. The device according to claim 29, wherein said front face further includes raised portions arranged between said second aperture and said edge.

31. The device according to claim 30, wherein said raised portions have a radial type arrangement.

32. The device according to claim 30, wherein said raised portions are concentric.

33. The device according to claim 18, wherein it includes positioning means for orienting the syringe in relation to the capping element when said syringe is inserted into said inner space.

34. The device according to claim 18, wherein said capping element includes a window for observing the level of liquid in the syringe when the latter is inserted inside said capping element.

35. A sleeve to be used as the capping element for implementing a device for reducing the pain associated with insertion of a needle of a syringe into a patient's skin according to claim 18.

36. A sleeve to be used as the capping element for implementing a device for reducing the pain associated with insertion of a needle of a syringe into a patient's skin according to claim 19.
